(19) United States
(12) Patent Application Publication
(10) Pub. No.: US 2022/0196654 A1
(43) Pub. Date: Jun. 23, 2022

(54) TESTING METHODS FOR DETERMINATION OF T2R PHENOTYPE AND APPLICATIONS THEREOF

(71) Applicant: Phenomune, LLC, The Woodlands, TX (US)

(72) Inventor: Henry P. Barham, Baton Rouge, LA (US)

(73) Assignee: Phenomune, LLC, The Woodlands, TX (US)

(21) Appl. No.: 17/693,928

(22) Filed: Mar. 14, 2022

Related U.S. Application Data
(63) Continuation-in-part of application No. 17/500,755, filed on Oct. 13, 2021.
(60) Provisional application No. 63/092,279, filed on Oct. 15, 2020.

(51) Int. Cl. G01N 33/566

(52) U.S. Cl. CPC G01N 33/566

(57) ABSTRACT

This disclosure provides methods of treating a human subject by stimulating chemosensory receptors, such as T2Rs, to increase level of phenotypic expression. Methods may include detecting phenotypic expression deficit by introducing a first agonist capable of first stimulating the chemosensory receptors by first binding thereto; detecting first stimulating; identifying a first deficit in relation to a first reference level, the first deficit being an instance of phenotypic expression deficit. Second stimulating with a second agonist may reduce the phenotypic expression deficit. Third stimulating with a therapeutic agonist may clear a respiratory illness condition by producing innate immune response.

### REFERENCE TO RELATED APPLICATIONS

This application claims priority from U.S. Non-provisional application Ser. No. 17/500,755 filed Oct. 13, 2021 titled TESTING METHODS FOR DETERMINATION OF T2R PHENOTYPE AND APPLICATIONS THEREOF, which is incorporated herein by reference in entirety and claims priority to U.S. Provisional Application No. 63/092,279, filed Oct. 15, 2020 titled TESTING METHODS FOR DETERMINATION OF T2R PHENOTYPE AND APPLICATIONS THEREOF, which is incorporated herein by reference in entirety.

### FIELD OF THE INVENTION

This disclosure relates to the characterization and stimulation of chemosensory receptors and applications of stimulating chemosensory receptors. More particularly, the disclosure relates to characterization and stimulation of T2R's and applications of stimulating T2Rs.

### BACKGROUND OF THE INVENTION

Chemosensory receptors are encoded by six families of genes including trace amine-associated receptors (TAAR), olfactory receptors (OR), vomeronasal receptor type 1 and 2 (V1R and V2R), and taste receptors type 1 and 2 (T1R and T2R). All of the chemosensory receptor proteins are G-protein coupled receptors. Chemosensory receptors may be expressed on the surface of solitary chemosensory cells (SCCs). Receptors belonging to Taste Receptor Family-1 subtypes 2 and 3 (T1R2/T1R3) detect sweet compounds such as glucose and sucrose. Taste Receptor Family-2 receptors (T2Rs) detect bitter taste compounds. Greater than fifty (50) T2Rs have been characterized. Stimulation of T2Rs activates, at least, the canonical taste signaling cascade involving phospholipase Cβ2 (PLCβ2) and transient receptor potential cation channel subfamily M member 5 [Nei, M., Niimura, Y. & Nozawa, M. The evolution of animal chemosensory receptor gene repertoires: roles of chance and necessity. Nature Reviews Genetics 9, 951-963 (2008)]. The aforementioned manuscript is hereby incorporated by reference in entirety.

T2Rs are genetically diverse, a phenomenon that helps to explain the wide variety of taste preferences both within and between cultures. Many individuals find various bitter foods to be detestable, while others do not have the same aversive response. This genetic variation of T2Rs is found on the tongue, but not exclusively. More recently, bitter and sweet receptors have been discovered in a variety of extra-oral tissues including the brain, thyroid, pancreas, testes and throughout the respiratory and gastrointestinal tracts.

A growing body of literature has suggested a role for bitter taste receptors (T2Rs) in sinonasal innate immunity. The literature suggests that genetically heritable differences in bitter taste receptors contribute to the pathogenesis of rhinosinusitis and upper respiratory tract infections. Extraoral bitter taste receptors present on ciliated mucosal cells and solitary chemosensory cells (SCC) are known to play a role in innate immunity and immune system activity. T2R receptors are also present in the airway and appear to play a key role in respiratory defense. Various reagents, compounds, and chemicals agonize T2Rs, causing the release of products that may contribute to clearing and killing pathogens in the human respiratory mucosa.

### BRIEF SUMMARY OF THE INVENTION

Need exists for reliable methods of treatment of human subjects that may include stimulating, and evaluating levels of phenotypic expression, of chemosensory receptors, such as T2Rs. Need exists for methods of treatment that include predicting susceptibility to, and clinical course of, microbial infections in subjects.

Embodiments include methods of treatment to treat a human subject by stimulating T2Rs to determine phenotypic expression. Embodiments include methods of treatment to treat a human subject by stimulating T2Rs to determine phenotypic expression deficit. Embodiments include methods of treatment to treat a human subject by stimulating T2Rs to improve phenotypic expression. Embodiments include methods of treatment to treat a human subject by stimulating T2Rs to provide reduced phenotypic expression deficit. Embodiments include methods of treatment of a human subject by stimulating T2Rs to provide reduced phenotypic expression deficit, and to further treat the human subject having the reduced phenotypic expression deficit by stimulating T2Rs to provide an elevated phenotypic expression including an innate immune response to address or clear a respiratory illness condition. Embodiments include methods to determine chemosensory receptor phenotype. Embodiments include methods for evaluating phenotypic expression of chemosensory receptors. Embodiments include methods for evaluating functionality of chemosensory receptors. Embodiments include test kits to perform chemosensory receptor phenotype testing. Embodiments include methods to improve receptor phenotypic expression of a human subject having reduced or other problematic phenotypes. In embodiments, such chemosensory receptors may be T2Rs. In embodiments, stimulation of T2Rs with one or more agonists may result in increased release of products that may improve innate immune response to address a respiratory condition, such as an upper respiratory infection. Such innate immune response, for example, may include responses to pathogens, microbial agents, bacterial pathogens, and viruses, and may include responses to SARS-CoV-2 and influenza. In embodiments, other chemosensory receptors may be stimulated.

In an embodiment a method of treatment may include detecting products released because of the stimulation of T2Rs. In an embodiment a test method may include determining a level of phenotypic expression of T2Rs. In embodiments, other chemosensory receptors may be stimulated. In an embodiment a method may include evaluating a level of phenotypic expression or level of functionality of T2Rs. In an embodiment a test kit may be configured for determining the phenotypic expression of T2Rs. In embodiments, methods of treatment and test kits may enable determining phenotype, or phenotypic expression, of other chemosensory receptors.

The above-mentioned shortcomings, disadvantages and problems are addressed herein, as will be understood by those skilled in the art upon reading and studying the following specification. This summary is provided to introduce a selection of concepts in simplified form that are further described below in more detail in the detailed description. This summary is not intended to identify key or essential features of the claimed subject matter.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Apparatus, systems, and methods of varying scope are described herein. These aspects are indicative of various non-limiting ways in which the disclosed subject matter may be utilized, all of which are intended to be within the scope of the disclosed subject matter. In addition to the aspects and advantages described in this summary, further aspects, features, and advantages will become apparent by reference to the associated drawings, detailed description, and claims.

### BRIEF DESCRIPTION OF DRAWINGS

The novel features believed characteristics of the disclosed subject matter will be set forth in the claims. The disclosed subject matter itself, however, as well as a mode of use, further objectives, and advantages thereof, will best be understood by reference to the following detailed description of illustrative embodiments when read in conjunction with the accompanying drawings, wherein:

FIG. 1 is a table showing clinical course of disease in relation to stratification by level of phenotypic expression of T2R38 for a plurality of human subjects in an exemplary embodiment;

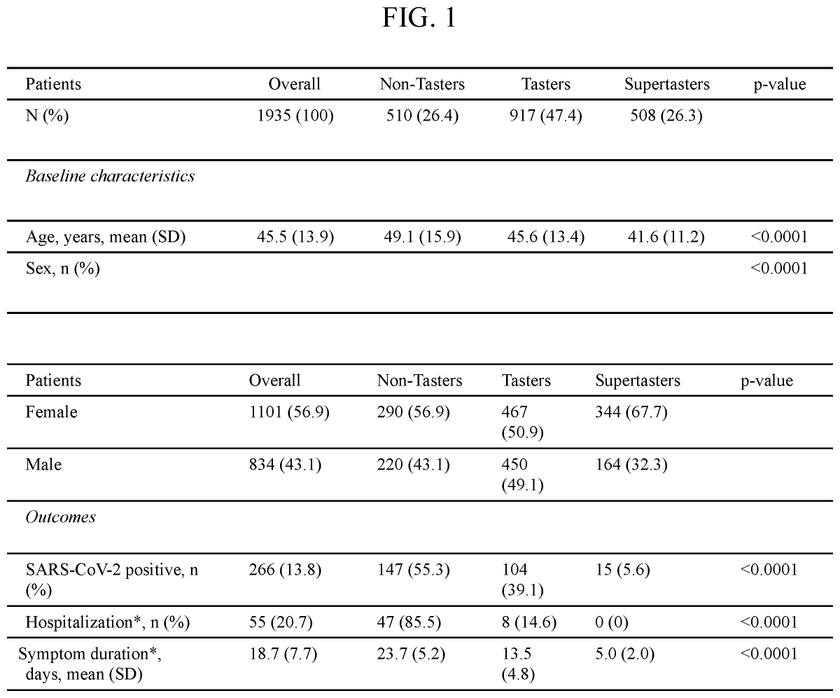

FIG. 2 is a table showing clinical outcomes in relation to stratification of patients by level of phenotypic expression of T2R38 for a plurality of human subjects in an exemplary embodiment shown generally in FIG. 1;

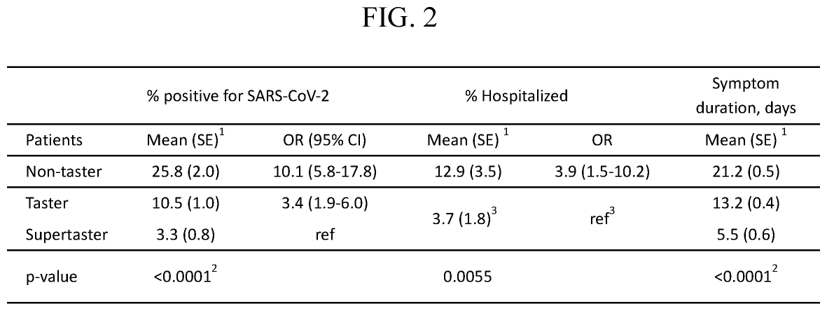

FIG. 3 is a table showing correlation of genotype stratified by number of functional genes in relation to phenotype stratified by level of phenotypic expression for a plurality of human subjects in an exemplary embodiment;

FIG. 4 is a table showing exceptions to the correlation shown generally in FIG. 3;

FIG. 5 is a simplified flow diagram illustrating aspects of a method 500 of treatment in an embodiment;

FIG. 6 is a simplified flow diagram illustrating aspects of a method 600 of treatment in an embodiment;

FIG. 7 is a simplified flow diagram illustrating aspects of method 700 of treatment in an embodiment;

FIG. 8 is a simplified flow diagram illustrating aspects of a method 800 of treatment in an embodiment;

FIG. 9 is simplified block diagram illustrating aspects of a test kit 900 in an embodiment;

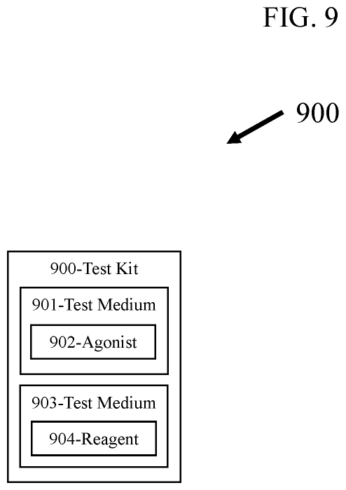

FIG. 10 is simplified block diagram illustrating aspects of a test kit 1000 in an embodiment;

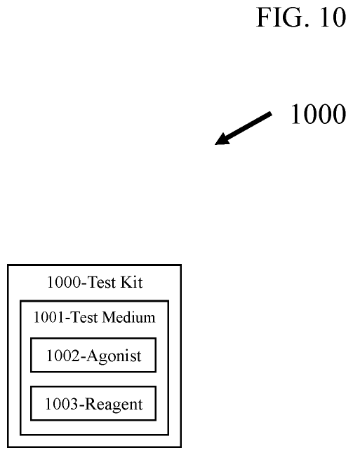

FIG. 11 is a simplified flow diagram illustrating aspects of a method 1100 of preparing a specimen, in an embodiment;

FIG. 12 is a simplified flow diagram illustrating aspects of method 1200 in an embodiment;

FIG. 13 is a simplified flow diagram illustrating aspects of a method 1300 in an embodiment;

FIG. 14 is a simplified flow diagram illustrating aspects of method 1400 in an embodiment;

FIG. 15 is a simplified block diagram illustrating aspects of a test kit 1500 in an embodiment; and

FIG. 16 is a simplified block diagram illustrating aspects of a test kit 1600 in an embodiment.

### DETAILED DESCRIPTION OF ILLUSTRATIVE EMBODIMENTS

In this detailed description, reference is made to the accompanying drawings which form a part hereof, and in which is shown by way of illustration specific embodiments which may be practiced. Reference now should be made to the drawings, in which the same reference numbers are used throughout the different figures to designate the same components. These embodiments are described in sufficient detail to enable those skilled in the art to practice the embodiments and disclosure. It is to be understood that other embodiments may be utilized, and that logical, mechanical, electrical, and other changes may be made without departing from the scope of the embodiments and disclosure. In view of the foregoing, the following detailed description is not to be taken as limiting the scope of the embodiments or disclosure.

The terminology used herein is for the purpose of describing particular embodiments only and is not intended to be limiting. As used herein, the singular forms “a”, “an”, and “the” are intended to include the plural forms as well, unless the context clearly indicates otherwise. It will be further understood that the terms “comprises” and/or “comprising” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof

It will be appreciated that for simplicity and clarity of illustration, where considered appropriate, reference numerals may be repeated among the figures to indicate corresponding or analogous elements. In addition, numerous specific details are set forth in order to provide a thorough understanding of the implementations described herein. However, it will be understood by those of ordinary skill in the art that the implementations described herein may be practiced without these specific details. In other instances, well-known methods, procedures and components have not been described in detail so as not to obscure the implementations described herein. Also, the description is not to be considered as limiting the scope of the implementations described herein.

The detailed description set forth herein in connection with the appended drawings is intended as a description of exemplary embodiments in which the presently disclosed apparatus and system can be practiced. The term “exemplary” used throughout this description means “serving as an example, instance, or illustration,” and should not necessarily be construed as preferred or advantageous over other embodiments.

For clarity, this disclosure is directed particularly in all aspects and embodiments to T2R's. In addition to T2R's, except where stated to the contrary, or implied to the contrary in the understanding of a person of ordinary skill, or where context would otherwise inform a person of ordinary skill, some embodiments disclosed herein also may find application in or with, or may include, methods, methods of treatment, assessments, evaluations, determining, treatments, uses, functionings, or stimulations of chemosensory receptors other than, or in addition to, T2R's. For clarity, as used throughout this document, the term “microbial infections” may refer to both infections caused by bacteria and by viruses, unless otherwise specified. For clarity, and without limitation, the word “product” refers to ancillary products of a reaction that are of interest. For clarity, and without limitation, the term “phenotypic expression” refers to observable characteristics of the human subject in relation to T2Rs. These characteristics may include, for example, taste perception of agonists that stimulate T2Rs, or the production of products because of the stimulation of T2Rs by agonists. Throughout this document, the terms “interacting” and “interaction” in reference to the reagent and the products released as a result of stimulating T2Rs refers to association, contact, and/or reaction, and may be cohesive, adhesive, repulsive forces, as applicable, as long as the interaction results in a detectable phenomenon. In embodiments, other chemosensory receptors may be stimulated.

The following examples are presented for purposes of illustration, and are not intended to impose limitations on the scope of embodiments disclosed herein.

### Example 1

In a clinical study of 1935 human patients who submitted to a taste test for the T2Rs, an expected distribution of approximately 25% strong reaction patients, 50% intermediate reaction patients, and 25% no-reaction patients was found. The study followed the 1935 patients for three months to observe their outcomes with the COVID-19 virus (SARS-CoV-2). A significant correlation was found between bitter taste receptor reaction and COVID-19.

Patients exposed to SARS-CoV-2 were enrolled from an outpatient clinical practice and an inpatient hospital from July 2020 through August 2020 and were followed prospectively until Sep. 30, 2020. All patients underwent phenotype taste testing, and each patient's SARS-CoV-2 status was confirmed via polymerase chain reaction (PCR), immunoglobulin M (IgM) and immunoglobulin G (IgG) testing to confirm absence of infection. Patients underwent study inclusion with phenotype taste testing and evaluation of lack of infection with SARS-CoV-2 via PCR (to exclude current infection), IgM and IgG (to exclude previous infection). Patients with evidence of active infection with SARS-CoV-2 via PCR at study commencement were excluded. Also excluded were patients with evidence of prior infection with SARS-CoV-2 via IgM and/or IgG at study commencement.

Levels of phenotypic expression of a T2R, specifically TAS2R38 (also referred to as T2R3 8), was determined via commercially available paper taste test strips to evaluate the genetically determined taste response phenotype of each subject. These tests included four separate taste test strips administered in the following order:

1. a control (no taste chemical),

2. phenylthiocarbamide (PTC, 3 to 5 μg),

3. thiourea, and

4. sodium benzoate.

The sodium benzoate taste test strip was used to help control for potential for global loss of taste associated with SARS-CoV-2. Patients with positive results to the control taste test strip were excluded from the study.

In the series of taste tests, the patients were each instructed to place the provided litmus paper taste test strip on their tongue until completely moistened, then the next litmus paper strip was provided in the order stated above. Sips of water were permitted between the application of each test strip. Patients were instructed to comment on the quality of taste they perceived and to comment on the intensity of the taste on a visual analog scale from 0 to 10, where 0 indicates no perception of taste and 10 indicates extremely intense taste quality perceived, as compared to the control taste test paper. Each patient was oriented to the scale with a verbal explanation prior to proceeding with the test.

All patients included in the study were categorized into 3 groups (supertasters, tasters, & non-tasters) via their level of phenotypic expression of T2R38. Scores of 0 and 1 to PTC were classified as non-tasters; scores of 2 to 8 to PTC were classified as tasters; and scores of 9 and 10 to PTC were classified as supertasters. Scores for thiourea were used to confirm the level of phenotypic expression of T2R38 primarily in tasters. A randomized subgroup of patients in the study underwent genotype analysis by Monell Chemical Senses Center, Philadelphia, Pa., USA, using Oragene® DNA collection kits and DNA Genotek® genetic testing kits to correlate phenotype.

Patients were followed until infection with SARS-CoV-2, as confirmed by PCR. Phenotype expression of T2R38 via the taste testing protocol described above was re-determined after infection and the results of both genotype and phenotype were correlated with clinical course and outcome of disease. Patients were stratified into more severe and less severe clinical course of disease according to need for hospitalization during their infected period. Patients requiring hospitalization for treatment comprise the more severe cohort. Outcomes related to severity of disease (days symptomatic and need for hospitalization) were assessed via medical records.

Statistical analyses were performed using SAS analytical software version 9.4. Unadjusted comparisons of baseline characteristics and outcomes were conducted via chi-square tests and analyses of variance. Logistic regression analyses and zero-inflated Poisson analysis were used to assess relationships between tasting phenotypes and outcomes; all models were adjusted for age and sex. All aspects of this study were reviewed and approved by the Baton Rouge General Institutional Review Board (IRB00005439).

Overall, 1935 subjects (mean age, 45.5 years; 56.9% female) participated in the study, with 510 (26.4%) being non-tasters, 917 (47.4%) tasters, and 508 (26.3%) supertasters, as determined by a subjective taste test (see Table 1). Results of the taste test suggest a decreasing level of phenotypic expression with increasing age (p<0.0001) among supertasters (mean age, 41.6 years), tasters (mean age, 45.6 years), and non-tasters (mean age, 49.1 years). Table 1 summarizes some of the baseline characteristics and outcomes of patients exposed to SARS-CoV-2.

During the study period, 266 (13.8%) patients tested positive for SARS-CoV-2 via PCR. Of these, 55 (20.7%) patients required hospitalization. Symptom duration among positive cases ranged from 0 to 48 days. Non-tasters were significantly more likely to test positive for SARS-CoV-2 (p<0.0001), to be hospitalized once infected (p=0.0055), and to be symptomatic for a longer duration (p<0.0001; see Table 2). Risk of infection and of symptom duration showed significant evidence of linear trends across the tasting phenotypes. Table 2 shows the relationships between taster classification, SARS-CoV-2 infection, and clinical consequences, where SE stands for standard error and CI stands for confidence interval.

### Example 2

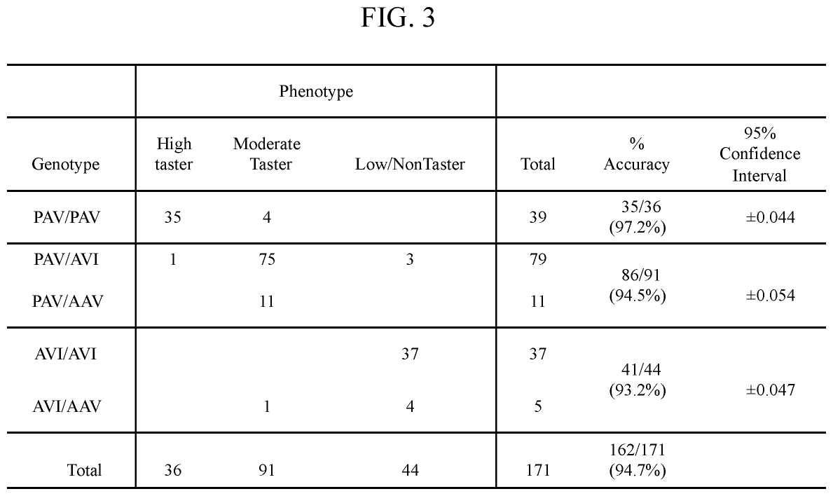

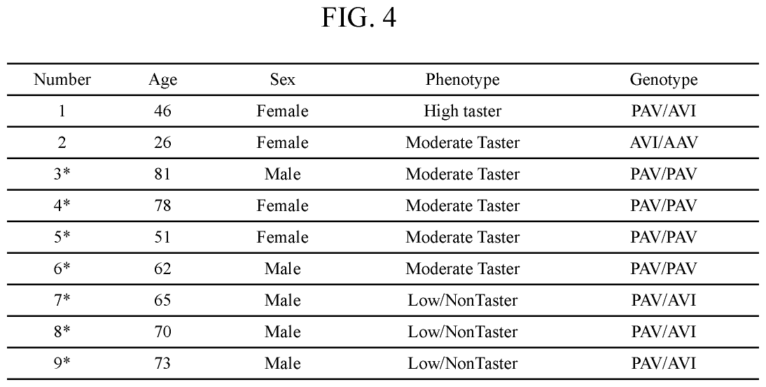

Aspects of an investigational device study performed at an outpatient clinical practice and inpatient hospital on 171 patients and health care workers are shown in FIGS. 3 and 4. All subjects were categorized into 3 groups (high tasters, moderate tasters, and low/non-tasters) via their level of phenotypic expression of T2Rs, tested using taste strip tests as described in Example 1. Subjects underwent genotype analysis to detect single nucleotide polymorphism (SNP) in the TAS2R38 gene. Three polymorphisms were genotyped using real-time PCR single nucleotide polymorphism genotyping assays (rs713598, rs1726866, and rs10246939). Correlation between level of phenotypic expression and genotype was conducted.

In the study, subjects were categorized as follows: subjects having two copies of the PAV allele were categorized as high taster, those with one copy of PAV allele as moderate taster, and finally, those with no PAV alleles in their genotype were classified as low/nontasters.

Participants with evidence of active infection with SARS-CoV-2 via PCR at study commencement were excluded. Participants with evidence of prior infection with SARS-CoV-2 via IgM and or IgG at study commencement were excluded. Participants were excluded from evaluation with positive results to the Control strip.

Statistical analyses were performed using SPSS v 22 (SPSS Statistics for Windows, version 22.0; IBM, Armonk, N.Y.). Descriptive data are presented as percentages and means±standard deviation (SD). Kendall's tau-B was used for ordinal values. Chi-squared analysis was used for relationships of nominal variables. Student t test (2-tailed) was used for comparisons of parametric data. Results were deemed significant with a p value of <0.05.

As shown in FIG. 3, one hundred seventy-one patients (171; 53.2% female) with a mean age of 41.56 years; were assessed with phenotype taste testing. All participants were categorized into 3 groups (high tasters, moderate tasters, and low/nontasters) via their level of phenotypic expression of T2R. Thirty-six (21.1%) were categorized as high tasters. Ninety-one (53.2%) were categorized as moderate tasters. Forty-four (25.7%) were categorized as low/nontasters (FIG. 3).

Referring to FIG. 3, genetic analysis of the 171 subjects revealed the PAV/PAV diplotype in 39 subjects, while 90 subjects were classified as heterozygotes (PAV/AVI & PAV/AAV) (79 PAV/AVI and 11 PAV/AAV). 42 subjects were categorized in the non-PAV containing group (37 AVI/AVI and 5 AVI/AAV), where AAV is alanine-alanine-valine.

Referring to FIG. 3, when evaluating the relationship between phenotype and genotype, phenotype showed 94.7% (162/171) accuracy in predicting genotype (p-value <0.01). The average age of the discordant subjects was 61.3. Rate of discordant results in high taster group was lower than the other two groups, with 35/36 (97.2%), with only 1 case testing phenotypically as a high taster but displayed a PAV/AVI genotype. The study showed discordant results in the moderate taster group (single PAV allele) higher than the other 2 groups, where 86/91 single-PAV carrying subjects (75 PAV/AVI and 11 PAV/AAV) tested phenotypically as moderate tasters (94.5%), with disagreement in 5 cases (4 PAV/PAV and 1 AVI/AAV). Lastly in the low/nontaster group, 41/44 subjects (37 AVI/AVI and 4 AVI/AAV) tested phenotypically in the low/nontaster group (93.2%), with 3 cases that displayed the PAV allele and still categorized phenotypically as a low/nontaster.

Referring to FIG. 4, in the nine subjects with discordant results, seven subjects (5 males and 2 females, average age 68.6 years) had a genotype of a higher group (PAV/PAV) but tested phenotypically in the lower group (moderate taster).

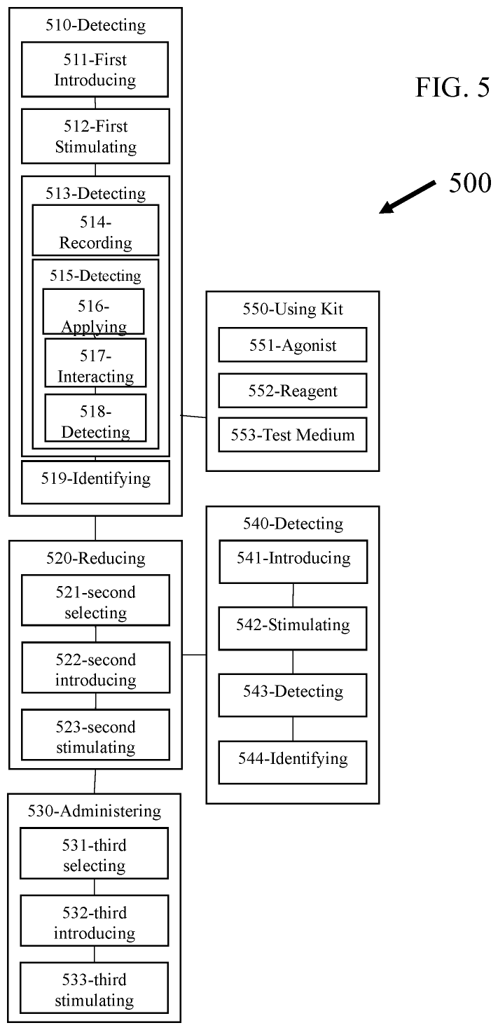

FIG. 5 is a simplified flow diagram of a method 500 of treating a human subject by stimulating chemosensory receptors to increase a level of phenotypic expression of the chemosensory receptors, which may be T2R's, in an exemplary embodiment. The chemosensory receptors, for example, initially may exhibit or present an original, unrehabilitated level of phenotypic expression. Method 500 may include primary detecting 510 a phenotypic expression deficit of the T2R's. Primary detecting 510 may include first introducing 511 to the chemosensory receptors a first agonist capable of first stimulating 512 the chemosensory receptors by first binding thereto.

Referring to FIG. 5, primary detecting 510 may further include first detecting 513 a first level of said first stimulating 512. In embodiments, the first detecting 513 the first level may include (i) recording discerned taste, (ii) detecting a released product, or (iii) both.

Referring to FIG. 5, in one such embodiment, the first detecting 513 the first level may include recording 514 taste discerned by the human subject after first stimulating 512. The discerned taste may be recorded, for example, from an answer to a query or indication of rating on a scale by the subject.

Referring to FIG. 5, in one embodiment, the first detecting 513 the first level may include secondary detecting 515 of a product released in relation to said first stimulating 512. In an embodiment, the secondary detecting 515 may further include applying 516 a reagent to interact with the released product to provide a detectable phenomenon. The secondary detecting 515 of the product may further include the applied reagent interacting 517 with the released product to provide a detectable phenomenon. The secondary detecting 515 of the product may further include tertiary detecting 518 of the detectable phenomenon produced by the applied reagent interacting 517 with the released product to provide the phenomenon.

Referring to FIG. 5, in an embodiment the primary detecting 510, of a phenotypic expression deficit of the T2R's, may include identifying 519 a first deficit of the first level in relation to a first reference level. In an embodiment, such reference level may include an average of a measurement of discerned taste levels for a group of subjects. The first deficit is an instance of the phenotypic expression deficit to be treated. In an embodiment, such reference level may include an average of a measurement of levels of products produced as a result of stimulation of T2Rs for a group of subjects. In an embodiment, the reference levels may vary as more subjects are added to the reference level group, such that the average changes. For clarity, in an embodiment, a reference level for a group of subjects, or a plurality of reference levels for categorized subgroups of subjects, may be determined for genotype of a T2R or plurality of T2R's, such as TAS2R38. In an embodiment, for example, a major group of patients may be categorized into three categories, or categorized subgroups, in relation to possible genotypes (which may be SNP's): (i) PAV/PAV, (ii) PAV/AVI, and (iii) AVI/AVI. A reference level may be determined for each categorized subgroup. For example, a reference level may be determined for each categorized subgroup in relation to indications of perceived taste by subjects, such as indications of perceived taste intensity on a numeric scale (such as a 1-10 numeric scale) when subjects are exposed to agonists. Such reference levels may be determined, for example, where a taste test is administered to patients in each of the categorized subgroups. Such a taste test, for example, may include administering paper test strips to the subject, and the test strips may include a control (no taste chemical), phenylthiocarbamide (PTC, 3 to 5 ug), thiourea, and sodium benzoate. The subjects may be instructed to provide a score, such as on a numeric scale of 1 to 10, wherein 0 indicates no perception of taste and 10 indicates extremely intense taste quality perceived, as compared to a control taste test paper. Statistical analysis may be performed to develop a reference level describing the taste perception characteristic, such as the mean, median, or mode for each subgroup, the major group, or both. In an embodiment, a reference level for a group of subjects, or a plurality of reference levels for categorized subgroups of subjects, may be determined for a characteristic correlated with genotype of T2R's. Such a correlated characteristic may include, for example, perceived taste response characteristic correlated with T2R genotype. A perceived taste response characteristic may include, for example, a supertaster characteristic, intermediate or normal taster characteristic, and a non-taster characteristic. The subjects categorized or stratified in relation to a taste response characteristic may be further correlated or stratified in relation to a history of actual clinical course of disease. In an embodiment, for example, such a history of actual clinical course of disease may be development of SARS-CoV-2. For example, in an embodiment, patients may be stratified by history of an actual clinical course of disease, into a more severe category and less severe category clinical course of disease, such as by need for hospitalization during a period of SARS-CoV-2 infection. Statistical analysis may be performed to develop a reference level, such as the mean, median, or mode, for each subgroup, the major group, or both. In an exemplary embodiment, determining a reference level may include: (a) categorizing a population of subjects for genotype of TAS2R38, such as by categorizing the population of subjects into three categories of possible SNPs, specifically: PAV/PAV, PAV/AVI, AVI/AVI; and, (b) for each of the three categories, determining a category reference level for all subjects having the genotype in the category. In (b), such determining category reference levels, for example, may include: (i) administering to each subject a taste test in relation to a plurality of agonists in series; (ii) recording for each subject taste perception results for the plurality of agonists administered in the taste test; and (iii) determining the category reference levels by developing for each category a statistical representation of the taste perception results for all subjects in said category. The statistical representation, for example, may include mean, median or mode of the taste perception results determined for each category. In an embodiment, for example, the plurality of agonists may include: a control (no taste chemical), phenylthiocarbamide (PTC, 3 to 5 ug), thiourea, and sodium benzoate, administered in series. In an embodiment, for example, the taste perception results may be determined by instructing each subject to indicate a score representing or indicating taste quality perceived by the subject, such as on a scale of 0 to 10, where 0 indicates no perception of taste, and 10 indicates extremely intense taste quality perceived, for administration of each agonist as compared to administration of the control taste test paper. In an embodiment, the taste test may be administered to each new subject, and the new patient may be assigned to one of the categories based on comparing the patient's indicated scoring of taste quality perceived with the statistical representation of taste perception results for each category, and making an inference that the new patient's genotype is in the category corresponding to the category statistical representation closest in value to the patient's assigned score of taste quality perceived for the plurality of agonists administered in the taste test.

As shown in FIG. 5, method 500 may include reducing 520 the phenotypic expression deficit. Such reducing 520 may include selecting 521 a second agonist in relation to the first deficit provided by the identifying 519. In some embodiments, reducing 520 may include such selecting a second agonist comprising a therapeutic agonist capable of interacting with the T2Rs to improve phenotypic expression and thus provide a reduced phenotypic expression deficit. Such reducing 520 may include second introducing 522 to the chemosensory receptors the selected second agonist capable of second stimulating 523 the chemosensory receptors by second binding thereto. The second stimulating 523 provides a reduced phenotypic expression deficit.

Referring to FIG. 5, method 500 may include second prime detecting 540 of the reduced phenotypic expression deficit of the T2R's. Such second prime detecting 540 may further include second detecting 543 a second level of the second stimulating 542. The second prime detecting 540 may include identifying 544 a second deficit of the second level in relation to a second reference level. The second deficit is an instance of the reduced phenotypic expression deficit to be treated. In some embodiments, the second reference level may be equal to the first reference level. In some embodiments, the second and first reference levels may differ. In an embodiment, such reference levels may include a statistical representation or statistical characteristic (“statistical representation”) such as an average, mean, mode, or measure of correlation, of a measurement of discerned taste levels for a reference population or group of subjects. In an embodiment, such reference levels may include a statistical representation of a measurement of levels of products produced as a result of stimulation of T2Rs for a reference population or group of subjects. In an embodiment, the reference levels may vary as more subjects are added to the reference population or group, such that value of the statistical representation, such as average, mean, mode or measure of correlation, changes in relation to changing composition of the reference population or group.

Referring to FIG. 5, method 500 may include such second prime detecting 540 of the second level by (i) recording discerned taste, (ii) detecting a released product, or (iii) both. In one such embodiment, the second prime detecting 550 the second level may include recording taste discerned by the human subject after second stimulating 523. The discerned taste may be recorded, for example, from an answer to a query or indication of rating on a scale by the subject. In one embodiment, the second prime detecting 550 the second level may include secondary detecting 543 of a product released in relation to said second stimulating 523. In an embodiment, the secondary detecting 543 may further include applying a reagent to interact with the released product to provide a detectable phenomenon. The secondary detecting 543 of the product may further include the applied reagent interacting with the released product to provide a detectable phenomenon. The secondary detecting 543 of the product may further include tertiary detecting of the detectable phenomenon produced by the applied reagent interacting with the released product to provide the phenomenon.

As shown in FIG. 5, method 500 may include administering 530, subsequent to reducing said phenotypic expression deficit, a therapeutic third agonist further agonizing the T2Rs characterized by said reduced phenotypic expression deficit to induce phenotypic expression comprising a therapeutic, innate immune response addressing or clearing a respiratory infection condition of the human subject. Such administering 530 thus may address or clear the unaddressed respiratory infection that is not addressed by an innate immune response of the T2Rs characterized by unrehabilitated phenotypic expression deficit that precedes second stimulating 523 to provide the reduced phenotypic expression deficit. As shown in FIG. 5, administering 530 may include third selecting 531 the therapeutic third agonist in relation to one of said reduced phenotypic expression deficit and said innate immune response, where the therapeutic third agonist is capable of stimulating the therapeutic, innate immune response to address or clear the respiratory infection condition. As shown in FIG. 5, administering 530 may include third introducing 532 to the chemosensory receptors said third agonist capable of third stimulating 533 the chemosensory receptors by third binding thereto to provide third stimulating 533 of the therapeutic innate immune response sufficient to address or clear the respiratory infection condition.

As shown in FIG. 5, method 500 may include using 550 a kit, or test kit, configured to enable performing steps of such method 500. Such kit, without limitation, may include an agonist 551, reagent 552, and test medium 553. The agonists 551 may include the first agonist, second agonist, and/or third agonist. Such kit may include one or more reagents 552 capable of interactions with products released by the T2R's in relation to stimulation of the T2R's by such agonists, to provide detectable phenomena. Such kit may include one or more or the test medium 553, which may contain and support the agonists, reagents, or both. In some embodiments, a kit may include: first agonist contained in a test medium, and second agonist contained in a test medium. In some embodiments, a kit may include: a reagent in a test medium, where the reagent is capable of interaction with a product released in relation to at least one of the first stimulating and second stimulating, said interaction providing a detectable phenomenon. In some embodiments, a kit may include: a first plurality of unique agonists contained in at least one test medium, and a second plurality of unique agonists contained in at least one test medium.

Referring to FIG. 5 illustrating method 500, in the first stimulating 512, the first agonist may comprise a first plurality of unique agonists. In embodiments where the first agonist comprises a first plurality of unique agonists, the method 500 may include recording 514 taste discerned by the human subject in relation to the first plurality of unique agonists in relation to the first stimulating 512. The first plurality of unique agonists may be applied in an order selected or determined for effectiveness in detecting products released in relation to stimulation of the T2R's. Such ordering, in embodiments, may include simultaneous application, or sequential application, of the first plurality of unique agonists as determined for effectiveness of detecting released products. In some embodiments, such detecting may include taste testing. In some embodiments, detecting may include detecting by a detection method. Such detection methods, for example, may include, visual detection of a color change of a test medium where a product is released. Such detection methods, for example, may include, a sensing method for detecting a released product, or detecting a phenomena of an interaction of a released product with a reagent. Such sensing methods, for example, may include: chemiluminescence sensing, electrochemical sensing, and optical sensing.

Referring to FIG. 5, in some embodiments the first agonist may include or consist of caffeine. In some embodiments the first agonist may include or consist of denatonium. In some embodiments the first agonist may include or consist of strychnine. In some embodiments the first agonist may include or consist of quinine. In some embodiments the first agonist may include or consist of a terpene. In some embodiments the first agonist may include or consist of phenylthiocarbamide. In some embodiments the first agonist may include or consist of thiourea. In some embodiments the first agonist may include or consist of sodium benzoate. In some embodiments the first agonist may include or consist of an agonist selected from the following: caffeine, denatonium, strychnine, quinine, a terpene, phenylthiocarbamide, thiourea, and sodium benzoate. In some embodiments the first agonist may include or consist of a first plurality of agonists selected from the following: caffeine, denatonium, strychnine, quinine, a terpene, phenylthiocarbamide, thiourea, and sodium benzoate. In an embodiment the first agonist may include or consist of a first plurality of agonists applied in a sequence as follows: (i) phenylthiocarbamide, (ii) thiourea, and (iii) sodium benzoate.

Referring to FIG. 5, in some embodiments the second agonist may include or consist of caffeine. In some embodiments the second agonist may include or consist of denatonium. In some embodiments the second agonist may include or consist of strychnine. In some embodiments the second agonist may include or consist of quinine. In some embodiments the second agonist may include or consist of a terpene. In some embodiments the second agonist may include or consist of phenylthiocarbamide. In some embodiments the second agonist may include or consist of thiourea. In some embodiments the second agonist may include or consist of sodium benzoate. In some embodiments the second agonist may include or consist of an agonist selected from the following: caffeine, denatonium, strychnine, quinine, a terpene, phenylthiocarbamide, thiourea, and sodium benzoate. In some embodiments the second agonist may include or consist of a second plurality of agonists selected from the following: caffeine, denatonium, strychnine, quinine, a terpene, phenylthiocarbamide, thiourea, and sodium benzoate. In an embodiment the second agonist may include or consist of a second plurality of agonists applied in a sequence as follows: (i) phenylthiocarbamide, (ii) thiourea, and (iii) sodium benzoate.

Referring to FIG. 5, in some embodiments the therapeutic third agonist may include or consist of caffeine. In some embodiments the third agonist may include or consist of denatonium. In some embodiments the third agonist may include or consist of strychnine. In some embodiments the third agonist may include or consist of quinine. In some embodiments the third agonist may include or consist of a terpene. In some embodiments the third agonist may include or consist of phenylthiocarbamide. In some embodiments the third agonist may include or consist of thiourea. In some embodiments the third agonist may include or consist of sodium benzoate. In some embodiments the third agonist may include or consist of an agonist selected from the following: caffeine, denatonium, strychnine, quinine, a terpene, phenylthiocarbamide, thiourea, and sodium benzoate. In some embodiments the third agonist may include or consist of a third plurality of agonists selected from the following: caffeine, denatonium, strychnine, quinine, a terpene, phenylthiocarbamide, thiourea, and sodium benzoate. In an embodiment the third agonist may include or consist of a third plurality of agonists applied in a sequence as follows: (i) phenylthiocarbamide, (ii) thiourea, and (iii) sodium benzoate.

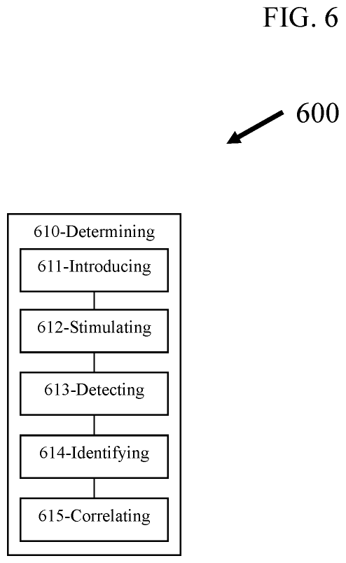

Illustrated in FIG. 6 is a method 600 of treating a human subject by stimulating chemosensory receptors to determine susceptibility to respiratory infection, in an embodiment. Method 600 may include determining 610 susceptibility to respiratory infection in relation to detecting phenotypic expression deficit. Determining 610 susceptibility may include first introducing 611 to the chemosensory receptors a first agonist capable of first stimulating 612 the chemosensory receptors by first binding thereto. Determining 610 susceptibility may include detecting 613 a first level of the first stimulating 612. Determining 610 susceptibility may include identifying 614 a first deficit of the first level in relation to a first reference level, said first deficit being an instance of said phenotypic expression deficit. Determining 610 susceptibility may include correlating 615 said phenotypic expression deficit to susceptibility to respiratory infection. In an embodiment, such reference levels may include a statistical representation or statistical characteristic (“statistical representation”) such as an average, mean, mode, or measure of correlation, of a measurement of discerned taste levels for a reference population or group of subjects. In an embodiment, such reference levels may include a statistical representation of a measurement of levels of products produced as a result of stimulation of T2Rs for a reference population or group of subjects. In an embodiment, the reference levels may vary as more subjects are added to the reference population or group, such that value of the statistical representation, such as average, mean, mode or measure of correlation, changes in relation to changing composition of the reference population or group.

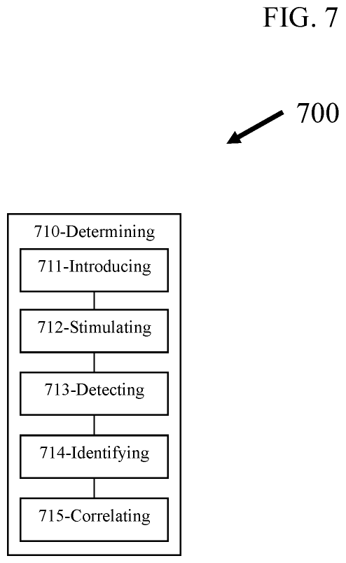

Illustrated in FIG. 7 is a method 700 of treating a human subject by stimulating chemosensory receptors to determine clinical course of respiratory infection, in an embodiment. Method 700 may include determining 710 clinical course of respiratory infection in relation to detecting phenotypic expression deficit. Determining 710 may include first introducing 711 to the chemosensory receptors a first agonist capable of first stimulating 712 the chemosensory receptors by first binding thereto. Determining 710 may include detecting 713 a first level of the first stimulating. Determining 710 may include identifying 714 a first deficit of the first level in relation to a first reference level, the first deficit being an instance of the phenotypic expression deficit. In an embodiment, such reference levels may include a statistical representation or statistical characteristic (“statistical representation”) such as an average, mean, mode, or measure of correlation, of a measurement of discerned taste levels for a reference population or group of subjects. In an embodiment, such reference levels may include a statistical representation of a measurement of levels of products produced as a result of stimulation of T2Rs for a reference population or group of subjects. In an embodiment, the reference levels may vary as more subjects are added to the reference population or group, such that value of the statistical representation, such as average, mean, mode or measure of correlation, changes in relation to changing composition of the reference population or group. Determining 710 clinical course of respiratory infection may include further correlating 715 phenotypic expression deficit to susceptibility to respiratory infection.

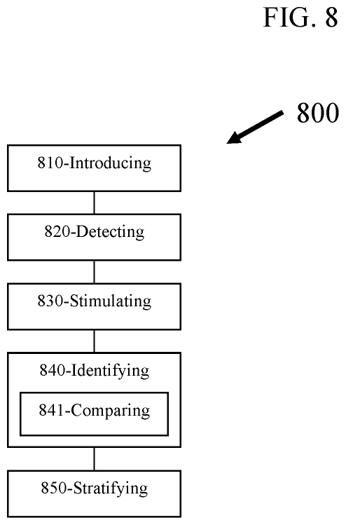

Illustrated in FIG. 8 is a method 800 of stratification of a patient into a category based on stimulating chemosensory receptors. Method 800 may include introducing 810 to the chemosensory receptors an agonist capable of stimulating 830 the chemosensory receptors when bound thereto. Method 800 may include detecting 820 a level of said stimulating 830. Method 800 may include identifying 840 a category by comparing 841 said level of said stimulating 830 to at least one reference level of stimulating. Method 800 may include stratifying 850 the patient into a category in relation to said comparing. Method 800 may include the reference level being at least one of the following: a high reference level for homozygous two functional alleles, a middle reference level for heterozygous one functional allele and one nonfunctional allele, and a low reference level for homozygous two nonfunctional alleles; and the category comprising inferred genotype. In an embodiment method 800 may include said reference level being at least one of the following: low level for low phenotypic expression, moderate level for moderate phenotypic expression, high level for high phenotypic expression; and said category comprising inferred level of phenotypic expression. In an embodiment method 800 may include said reference level being at least one of the following: low capacity for treatment by stimulating chemosensory receptors, moderate capacity for treatment by stimulating chemosensory receptors, and high capacity for treatment by stimulating chemosensory receptors; and the category being a selected one of the following: inferred capacity for treatment, inferred treatment outcome prediction, and course of treatment. In an embodiment method 800 may include said reference level being at least one of the following categorizations: Super tasters, Tasters, and Non-Tasters; and the category being a selected one of the following: inferred risk of infection, and inferred course of disease prediction. In an embodiment method 800 may include said reference level being at least one of the following categorizations: Super tasters, Tasters, and Non-Tasters; and the category being a level of functionality of chemosensory receptors. In an embodiment, such reference levels may include an average of a measurement of discerned taste levels for each stratified group of subjects. In an embodiment, such reference levels may include an average of a measurement of levels of products produced as a result of stimulation of T2Rs for a stratified group of subjects. In an embodiment, such reference levels may include a statistical representation or statistical characteristic (“statistical representation”) such as an average, mean, mode, or measure of correlation, of a measurement of discerned taste levels for a reference population or group of subjects. In an embodiment, such reference levels may include a statistical representation of a measurement of levels of products produced as a result of stimulation of T2Rs for a reference population or group of subjects. In an embodiment, the reference levels may vary as more subjects are added to the reference population or group, such that value of the statistical representation, such as average, mean, mode or measure of correlation, changes in relation to changing composition of the reference population or group. In an embodiment method 800 may include correlating level of phenotypic expression with level of function of T2Rs. In an embodiment method 800 may include correlating level of phenotypic expression with risk of infection from SARS-CoV-2. In an embodiment method 800 may include correlating level of phenotypic expression with clinical course of disease, SARS-CoV-2. In an embodiment method 800 may include correlating level of phenotypic expression with innate immune fitness.

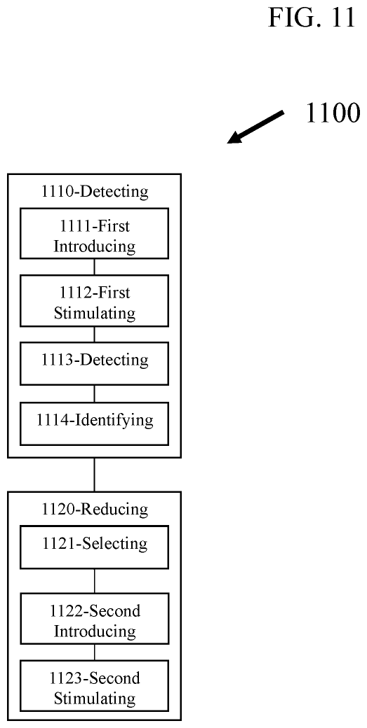

Illustrated in FIG. 11 is a method 1100 of preparing a specimen for treatment by agonizing stimulation. Method 1100 may include primary detecting 1110 phenotypic expression deficit. Primary detecting 1110 may include first introducing 1111 to chemosensory receptors a first agonist capable of first stimulating 1112 the chemosensory receptors by first binding thereto. Primary detecting 1110 may include secondary detecting 1113 a first level of said first stimulating 1112. Primary detecting 1110 may include identifying 1114 a first deficit of said first level in relation to a first reference level. In an embodiment, such reference levels may include a statistical representation or statistical characteristic (“statistical representation”) such as an average, mean, mode, or measure of correlation, of a measurement of discerned taste levels for a reference population or group of subjects. In an embodiment, such reference levels may include a statistical representation of a measurement of levels of products produced as a result of stimulation of T2Rs for a reference population or group of subjects. In an embodiment, the reference levels may vary as more subjects are added to the reference population or group, such that value of the statistical representation, such as average, mean, mode or measure of correlation, changes in relation to changing composition of the reference population or group. The first deficit is an instance of the phenotypic expression deficit. Method 1100 may include reducing 520 said phenotypic expression deficit to prepare for treatment by agonizing stimulation. Reducing 520 may include selecting 521 a second agonist in relation to said first deficit. Reducing 520 may include second introducing 522 to the chemosensory receptors said second agonist capable of second stimulating 523 the chemosensory receptors by second binding thereto. The second stimulating 523 provides a reduced phenotypic expression deficit.

In an embodiment, a method for treatment may comprise stimulating T2Rs with one or more agonists. In test methods for determining a level of phenotypic expression of T2Rs, the stimulating may involve exposing at least a portion of tissue of the human subject to one or more agonists. In some embodiments, stimulating may involve exposing a portion of oral or nasal tissue to agonists that stimulate T2Rs. In embodiments, other chemosensory receptors may be stimulated.

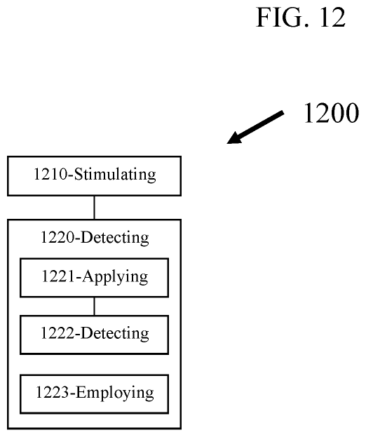

Illustrated in FIG. 12 is a method 1200 in an embodiment. Method 1200 may comprise stimulating 1210 T2Rs with at least one agonist. Method 1200 may comprise detecting 1220 one or more products released because of the stimulation of T2Rs. In an embodiment, method 1200 may comprise i) applying 1221 one or more reagents which interact with one or more of the products released because of the stimulation of the T2Rs, and ii) detecting 1222 the interaction of the reagent with one or more products, optionally by employing chemiluminescence, an electrochemical sensor, or an optical sensor, to detect the interaction of one or more reagents with one or more of the products. Method 1200 may comprise sensing 1223 chemiluminescence, an electrochemical property, or an optical property to detect one or more of the products released because of the stimulation of the T2Rs. In embodiments, other chemosensory receptors may be stimulated.

Detection of the interaction of the reagent with one or more products released because of the stimulation of T2Rs may comprise visual detection, taste, or both.

In embodiments, methods may comprise stimulating T2Rs with one or more agonists, and detecting one or more products released because of the stimulation of T2Rs by one of the detecting methods described herein. In embodiments, other chemosensory receptors may be stimulated.

In some methods, the agonists may be separately applied prior to applying the reagent which interacts with one or more products released because of stimulation of T2Rs. In this test method, the reagent in one embodiment is contained in or on a test medium. In other methods embodying an aspect of this invention, the agonists and the reagents which interact with one or more of the products released because of stimulation of T2Rs are contained in or on one or more test media. In other embodiments, an agonist and a reagent are present on or in the same test medium. In embodiments, other chemosensory receptors may be stimulated.

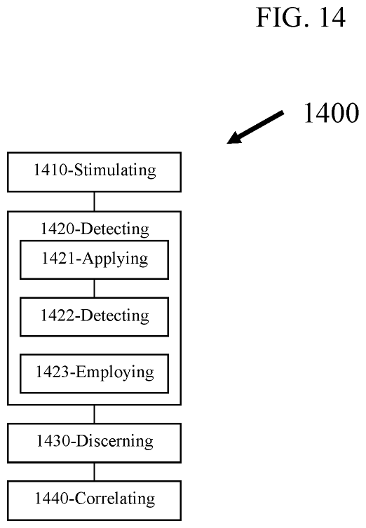

Illustrated in FIG. 14 is a method 1400 for evaluating a level of expression or functionality of T2Rs. Method 1400 may comprise stimulating 1410 T2Rs with one or more agonists, and detecting 1420 one or more products released because of the stimulation of the T2Rs. In an embodiment, a method may comprise: i) applying 1421 one or more reagents which interact with one or more of the products released because of the stimulation of the T2Rs. In an embodiment, a method may comprise: ii) detecting 1422 the interaction of the reagent with one or more products, optionally via chemiluminescence, an electrochemical sensor, or an optical sensor. In an embodiment, a method may comprise steps i) and ii) as disclosed in the preceding statements, in an ordered combination. A method may comprise employing 1423 chemiluminescence, an electrochemical sensor, or an optical sensor to detect one or more of the products released because of the stimulation of the T2Rs. In an embodiment, a method may include discerning 1430 a level of products produced because of the stimulation of the T2Rs that may be indicative of level of expression or functionality of the T2Rs. In embodiments, level of expression or functionality of the T2Rs may be correlated 1440 to a level of phenotypic expression. In embodiments, other chemosensory receptors may be stimulated.

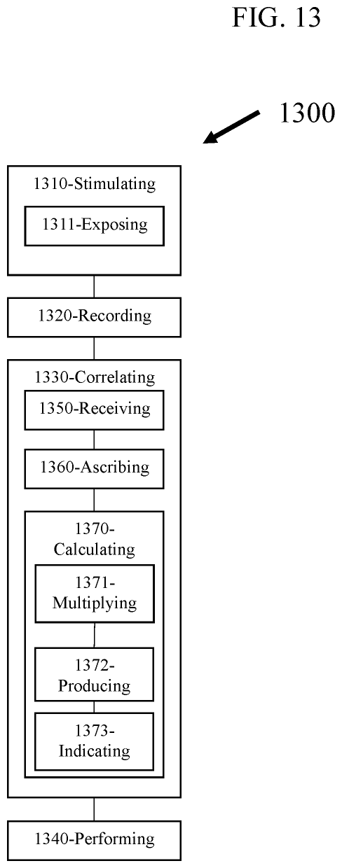

Illustrated in FIG. 13 is a method 1300. In test method 1300, stimulating 1310 T2Rs may comprise exposing 1311 at least a portion of tissue to one or more agonists. An embodiment may also include a method of recording 1320 a discerned level of taste perception by the human subject after the stimulation, and correlating 1330 the discerned level of taste perception by the human subject to a level of phenotypic expression of the T2Rs. In embodiments, other chemosensory receptors may be stimulated.

In embodiments, a test method for determining a level of phenotypic expression may be repeated two or more times. In embodiments this test method may be repeated one or more times using a therapeutic agonist. In embodiments the repeating of test methods may be used to obtain a data set. In embodiments, trend analysis may be performed on the data set.

When repeating the test method for determining a level of phenotypic expression is performed two or more times, the repeating may be performed at regular time intervals. The time intervals each may be 8 hours, daily, weekly, biweekly, monthly, bimonthly, semiannually, annually, or biannually. In an embodiment, such repeating two or more times may be performed at irregular time intervals.

In an embodiment of the test method for determining a level of phenotypic expression, the stimulating by each of one or more different agonists may be sequential. Recording of each discerned level of taste perception by the human subject may occur after each stimulation. Discerned levels of taste perception may be correlated to a level of phenotypic expression of the T2Rs. In embodiments, the correlating may comprise employing a computer processor programmed with machine-readable instructions that may cause the computer processor to a) receive 1350 and store the discerned levels of taste perception with respect to each agonist, b) ascribe 1360 a weighting to each of the agonists according to their known stimulation of T2Rs, and c) calculate 1370 a weighted taste perception from the discerned level of taste perception by multiplying 1371 the ascribed weighting and discerned level of taste perception for each agonist applied, then produce 1372 an aggregated, weighted level of taste perception which indicates 1373 the level of phenotypic expression. In embodiments, other chemosensory receptors may be stimulated.

In embodiments including application of a reagent, an agonist may be separately applied prior to applying the reagent. In an embodiment, when the product with which the reagent interacts decays relatively quickly (e.g., nitric oxide), the reagent may be applied during or soon (e.g., less than a minute) after the agonist is applied. In embodiments, a test medium containing the reagent that interacts with one or more products may be applied without use of an agonist. An embodiment may be, for example, when a patient has an infection known to stimulate T2Rs that causes the release of product(s) with which the reagent will interact. In an embodiment where a test medium contains reagent that interacts with one or more products, an agonist may be used to ensure a measurable response. In embodiments, other chemosensory receptors may be stimulated and the same principles applied.

In some methods, the use of two or more different agonists to stimulate the T2Rs may improve the accuracy of the determination of the level of phenotypic expression, compared to the use of one agonist. Similarly, the use of more agonists may improve the accuracy of determination of the level of phenotypic expression, compared to the use of fewer agonists. In embodiments, other chemosensory receptors may be stimulated. In an embodiment, for example, use of one agonist may provide a level of accuracy of about 50% to about 60% for determining phenotypic expression. In an embodiment, for example, use of two different agonists may provide a level of accuracy of about 70% to about 80% for determining phenotypic expression; In an embodiment, for example, use of three different agonists may provide a level of accuracy of about 94% or more for determining phenotypic expression. In an embodiment, for example, using four or more agonists may not significantly improve accuracy compared to use of three agonists. In an embodiment, for example, use of two different agonists may provide a suitable level of accuracy for determining level phenotypic expression. In an embodiment, for example, use of three different agonists may provide a more suitable level of accuracy for determining level of phenotypic expression, compared to use of two different agonists. In embodiments, a control test run may be performed using a blank that does not contain an agonist. In embodiments, other chemosensory receptors may be stimulated and the same principles applied.

In embodiments, stimulation of T2Rs of a human subject by one or more agonists may occur, for example, via topical, nasal, oral, gastrointestinal application of the agonists. In embodiments, the stimulation may comprise exposing tissue to one or more agonists. In embodiments, other chemosensory receptors may be stimulated. Suitable test media may vary with the nature of the agonist. Suitable test media may vary with the nature of the reagent present therein or thereon. Suitable test media may vary with the location of application of the agonist. Suitable test media may vary with the location of application of the reagent present therein or thereon. In an embodiment where the agonist is orally applied, test media may include paper strips, tongue depressors, and cotton swabs. In embodiments, paper strips may be used as the test medium.

Embodiments of test methods of this disclosure may comprise using one or more agonists to stimulate bitter taste receptor(s) (T2Rs). In embodiments, a test method may include using such an agonist for stimulation of T2Rs, wherein such agonist may include, for example, caffeine, denatonium (salts), strychnine, quinine, phenylthiocarbamide (PTC), and thiourea. In embodiments, two or more agonists may be used. In embodiments, stimulation of T2Rs by the agonist is perceivable as taste. In embodiments, stimulation of T2Rs by the agonist may lead to a taste, taste level, or discerned taste in a sensory system which includes the T2Rs. In embodiments, stimulation of T2Rs by the agonist may produce a signal response, perceived as taste by the human subject, in a sensory system which includes the T2Rs. In embodiments, a taste, taste level, or discerned taste produced in a sensory system may be indicated or identified by a human subject responsive to a query. In embodiments, agonists for therapeutic use may be different from agonists used to stimulate T2Rs for testing purposes. In embodiments, some agonists for therapeutic use may be identical to agonists used to stimulate T2Rs for testing purposes. In embodiments, therapeutic agonists for stimulation of T2Rs may include vitamins, nitric oxide releasers, caffeine, denatonium, strychnine, quinine, xylitol, grapefruit seed extract or naringenin, or a terpene. In embodiments, therapeutic agonists may be present in edible foods such as, for example, broccoli. In embodiments, two or more therapeutic agonists may be used. While other agonists for T2Rs are known, the agonists listed herein are exemplary of agonists which may be inexpensive and readily available. The agonists listed herein are not intended to be an exhaustive list of all agonists that may be used for the methods in this disclosure. In embodiments, other chemosensory receptors may be stimulated and the same principles applied.

Stimulation of T2Rs may cause the production of products such as, for example, antimicrobial peptides, nitric oxide or both. It will be understood that the preceding listing of exemplary products is not exhaustive, and other products may be produced. In an embodiment, a reagent may interact with one or more products released because of the stimulation of T2Rs. In an embodiment, for example, a reagent may interact with the antimicrobial peptides or nitric oxide, or other products. In an embodiment, a suitable reagent may comprise, for example, a Griess reagent. In an embodiment, T2Rs may cause the release of products because of stimulation by agonists. In an embodiment, a reagent interaction with one or more products may indicate functionality of the T2Rs. In an embodiment, reagent interaction with one or more products may be observed by qualitative or quantitative methods.

In embodiments a reagent that interacts with one or more products produced because of the stimulation of T2Rs may be applied before applying an agonist to determine a baseline. In embodiments, other chemosensory receptors may be stimulated and the same principles applied.

One or more reagents which interact with one or more of the products released because of the stimulation of T2Rs may include combinations of reagents to provide a detectable phenomenon. In embodiments, more than one reagent may be necessary to provide a detectable phenomenon. In some embodiments, for example, the detectable phenomenon may be a detectable color change. The color change may be determinable by spectroscopy or visually detectable (visible to human eyes of normal visual acuity). In embodiments, other chemosensory receptors may be stimulated.

In embodiments, stimulation of T2Rs by the agonist may produce a signal response, perceived as taste by the human subject, in a sensory system. In embodiments, a discerned level of taste perception by the human subject, that may occur in relation to a signal response produced because of the stimulation of T2Rs by an agonist, may refer to intensity or strength of the taste, or an absence of taste, as perceived by the human subject. For example, bitterness may be perceived by the human subject as a result of stimulation of T2Rs by an agonist. In an embodiment, the discerned level of taste perception as perceived by the human subject may be correlated to a level of phenotypic expression. In an embodiment, correlation of discerned level of taste perception to a level of phenotypic expression may be based on known levels of stimulation of T2Rs caused by the agonist used to stimulate the T2Rs. In an embodiment, known levels of T2R stimulation by agonists may be previously recorded levels of taste perception from the same or other human subjects. In an embodiment, known levels of T2R stimulation by agonists may be previous levels of product release as a result of T2R stimulation by the agonist of the same or other subjects. In embodiments, other chemosensory receptors may be stimulated and the same principles applied.

An embodiment may include using a detected level of products produced because of the stimulation of the T2Rs as a measure of expression. An embodiment may include using a detected level of products produced because of the stimulation of the T2Rs as a measure of functionality. An embodiment may include using a detected level of products produced because of the stimulation of the T2Rs as a measure of expression and functionality. Level of expression or functionality may be indicative of phenotypic expression. The detected level of products produced because of the stimulation of T2Rs may be indicative of, or correlated to, the level of phenotypic expression. In embodiments, other chemosensory receptors may be stimulated and the same principles applied.

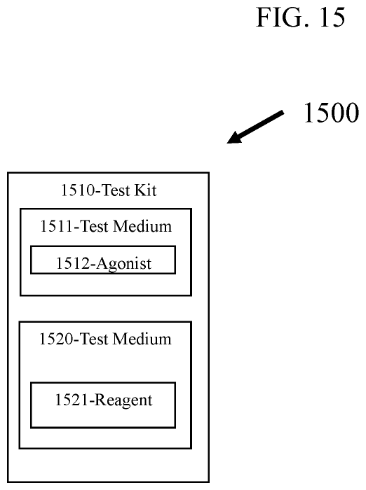

FIG. 15 illustrates a method 1500. In method 1500 a test kit is provided. The test kit may comprise at least one test medium 1511 containing one or more agonists 1512 for T2Rs or other chemosensory receptors, and at least on test medium 1520 containing one or more reagents 1520 which interact with one or more of the products released because of the stimulation of T2Rs or other chemosensory receptors.

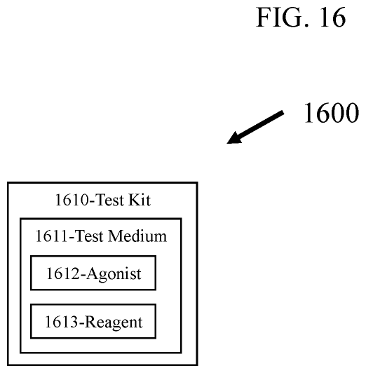

FIG. 16 illustrates method 1600. In method 1600, a test kit may include at least one test medium 1611 containing, or coated in, one or more agonists 1612 for T2Rs or other chemosensory receptors. In some embodiments, a test medium may comprise (a) one or more agonists 1612 for T2Rs or other chemosensory receptors, (b) one or more reagents 1613 which interact with one or more of the products released because of the stimulation of the T2Rs, or both (a) and (b). The interaction of the reagent with one or more products released because of the stimulation of the T2Rs may provide a detectable phenomenon that may be, for example, a color change.

In embodiments, the test medium may contain both one or more agonists for T2Rs and one or more reagents which interact with one or more of the products released because of the stimulation of T2Rs. In embodiments, a test medium may contain one agonist and one reagent. In embodiments, other chemosensory receptors may be stimulated and the same principles applied.

In an embodiment, a test kit may contain one or more paper strips, which paper strips may contain different agonists. In an embodiment, paper strips containing the same agonist and/or reagent in the same amount(s) may be stored in a common container. Paper strips with different agonists and/or reagents or different amounts thereof, may be stored in different containers. In an embodiment, a test kit may include one or more test strips of each type. Such one or more test strips of each type may include different numbers of test strips of different types, or equal numbers. The number or types of test strips and the number of each type of test strip may vary depending on the treatment program or protocol (what is being monitored, length of treatment, and frequency of testing). Optionally, a test kit may have a unique identifier.

In embodiments, methods and test kits having two or more agonists, two or more reagents, or both, may more accurately indicate an individual's susceptibility and clinical course to microbial infections, inflammatory disease and other diseases or symptoms that correlate with T2R or any chemosensory receptor stimulation, compared to the use of a single agonist, single reagent, or both.

The TAS2R genes may localize primarily to chromosomes 7 and 12, each with functional and non-functional polymorphisms. The correlation between taste receptor genetics and function and its potential role in sinusitis presentation and outcome may have been first characterized for the bitter taste receptor T2R38. The gene for this bitter receptor, TAS2R38, has at least two prevalent allele polymorphisms that correlate with taste receptor function. At least one functional allele may be characterized by a position 49 proline, position 262 alanine, and position 296 valine (known as a PAV genotype). At least one nonfunctional allele may be characterized by alanine, valine, and isoleucine at the respective positions (AVI genotype). Individuals who are homozygous for two functional alleles may be considered super-tasters who may be able to perceive bitter compounds such as propylthiouracil. Those who are homozygous for two nonfunctional alleles may be considered non-tasters who may be unable to perceive tastes after applying agonists, like, for example, propylthiouracil. Those who are heterozygous may have moderate taste perception after applying agonists, like, for example, propylthiouracil.

Genetic and/or environmental effects may contribute, albeit may be to different degrees, to phenotypic expression of T2Rs and other chemosensory receptors throughout life. Regarding bitter taste receptors (T2Rs), changes in gene expression in the development phase or hormonal influences around the time of puberty, may account for different levels of T2R stimulation, leading to different phenotypes such as, for example, reduced taste perception and reduced release of products because of T2R stimulation. Chemosensory receptor function may vary among individuals due to, for example, genetic polymorphisms. While only a few of these polymorphisms may have well-documented phenotypic effects, hundreds of T2R polymorphisms and several T1R polymorphisms have been noted in humans. Other polymorphisms of any chemosensory receptor may lead to different phenotypes that could be useful in the scope of this disclosure. The most well-known and well characterized example is the bitter receptor T2R38. The TAS2R38 gene encoding T2R38 has at least two common polymorphisms, one encoding a functional receptor and one encoding a nonfunctional receptor. The differences in the resulting proteins are at least at amino acid positions 49, 262, and 296. At least one functional T2R38 receptor contains proline (P49), alanine (A262), and valine (V296) residues while the nonfunctional T2R38 contains at least alanine (A49), valine (V262), and isoleucine (1296) at these positions, respectively. At least the loss of the valine at the third position in the AVI variant may prevent receptor activation.

These polymorphisms may be distributed in a nearly Mendelian ratio in Caucasian populations. Homozygous AVI/AVI individuals (approximately 30% frequency in Caucasian populations) may be “non-tasters” for the T2R38-specific agonists' phenylthiocarbamide (also known as phenylthiourea) and propylthiouracil. Homozygous PAV/PAV individuals (approximately 20% frequency in Caucasian populations) may be termed “super tasters” for these agonists because they may perceive them as intensely bitter, while AVI/PAV heterozygotes may have varying intermediate levels of taste. In some individuals, the T2R38 receptor contains alanine at position 49 (A49), alanine at position 262 (A262), and valine at position 296 (V296). Furthermore, individual differences in the expression of the PAV (Proline, Alanine, Valine) haplotype among heterozygotes may account for the variation in bitter taste perception. Consequently, a continuum of intermediate levels of responsiveness may separate the insensitive phenotype from the hypersensitive phenotype. These same principles may be useful in the application of other chemosensory receptors.

Genetic variations in taste receptor functionality may correlate with disease severity in chronic rhinosinusitis (CRS) and other diseases, disorders, or symptoms. Such correlation has been characterized, and may be further characterized, for patients who are homozygous for the non-functional variant of T2R38. Such patients may be more likely to require surgical intervention for CRS, and more likely to develop a Gram-negative infection. Recent work has shown that phenotypic taste tests with denatonium, a broad T2R agonist, and sucrose, a T1R2/3 agonist, may reflect clinical disease status in CRS and partially stratify control subjects and CRS patients. It is thought that patients with CRS possess hypo-responsive bitter taste receptors, rating denatonium as less bitter than controls, while also possessing hypersensitive sweet taste receptors, which may compound the reduced antimicrobial response to sinonasal pathogens.

Bitter taste receptor phenotype may correlate with clinical course following infection. Each individual's susceptibility to bacterial infections, viral infections, and inflammatory diseases may be predicted by the level of phenotypic expression of T2Rs or other chemosensory receptors. While one is able to evaluate for receptor functionality via genetic analysis, the level of phenotypic expression may better predict the clinical course of infection. Prior studies have shown that levels of phenotypic expression may decrease as humans age. Therefore, genotype may not be predictive of disease susceptibility when genotype is not predictive of phenotypic expression. The currently available taste tests to assess the level of phenotypic expression, which may show subjective results, are often misinterpreted because they do not rely on a plurality of taste tests together.

T2Rs line the tongue, but are also expressed on the surface of ciliated epithelial cells of the upper respiratory tract. T2Rs may make up part of the innate immune system and the function of specific T2Rs may be genetically determined with almost equal prevalence of functional and non-functional genotypes in the population. Recent work demonstrates that the NO-producing T2R response may be found in ciliated cells. Production of antimicrobial peptides may be driven only by T2Rs on SCCs.

In the airway, taste receptors may be present on a variety of cell types and have been shown to mediate several components of innate immune defense. For example, ciliated sinonasal epithelial cells express T2R38 and respond to PTC and acyl-homoserine lactones, bitter compounds released by gram-negative bacteria such as Pseudomonas aeruginosa. Activation of T2R38 triggers an increase in intracellular calcium (Ca²⁺) yielding stimulation of NO synthase with resultant production of intracellular NO. The NO, through cyclic GMP, increases ciliary beat frequency (CBF) and diffuses into the mucus layer where it has direct bactericidal activity.

One subset of T2Rs, when activated, may stimulate the respiratory epithelium to generate NO, while a second subset of T2Rs expressed on solitary chemosensory cells (SCCs), may stimulate release of antimicrobial peptides. Both T2R-mediated pathways may be integral to the upper airway innate immune defense system. There may be other pathways of chemosensory receptors that, when stimulated, lead to the generation of products that have positive therapeutic effects on fighting infections, reducing symptoms, or treating disorders.

The TAS2R38 allelic makeup may directly correlate with ability to generate NO in response to T2R38 stimulation. The TAS2R38 allelic makeup directly correlates with ability to clear P. aeruginosa from explanted sinonasal ciliated cells.

Stimulation of ciliated nasal epithelial cells with agonists may induce release of at least nitric oxide (NO). Release of NO from epithelial cells may reduce the growth of pathogenic bacteria among other positive effects.

With regard to whether NO may be helpful in the treatment of SARS-CoV-2 infection, SARS-CoV replication may be inhibited by NO when supplied by the NO donor molecule S-nitroso-N-acetylpenicillamine. NO may also prevent maturation of the viral S protein of SARS-CoV through inhibition of palmitoylation that results in reduced binding and viral fusion, leading to reduced capacity for viral entry. NO may also prevent or treat SARS-CoV-2 infection by other mechanisms.

NO may be able to inhibit viral replication of numerous viruses including the severe acute respiratory syndrome coronavirus (SARS-CoV and SARS-CoV-2). As the nasal airway is a primary portal of entry, SARS-CoV-2 infection via the nasal route may be suppressed by agonist-induced NO production from ciliated nasal epithelial cells. Agonist induced NO production may lead to decreased incidence and severity of SARS-CoV-2 infection.

Improvement of the innate immune response within the nasal cavity has potential to reduce the burden of chronic rhinosinusitis (CRS), along with other viral upper respiratory inoculations. It has been shown that stimulation of nasal epithelial cells with phenylthiocarbamide (PTC), another T2R agonist, induces transmembrane calcium fluxes that correlate with NO release. This stimulation is associated with reductions in bacterial growth. Exposure to NO has been shown to inhibit replication for many DNA and RNA viruses including hantavirus and the murine hepatitis coronavirus among others.

SCCs, rare epithelial cells that express both T1R2/3 and T2R receptors, also respond to bitter compounds secreted by bacteria in the upper airway. Stimulation of T2Rs on the surface of human SCCs by the bitter agonist, for example, denatonium elicits a calcium response that spreads via gap junctions to neighboring epithelial cells, triggering a release of preformed stores of antimicrobial peptides.

In some embodiments, a method or steps thereof may be repeated one or more times. In embodiments, performing a method or step thereof as herein disclosed may provide a data set. The methods and test kits as disclosed herein may be used at regular time intervals, as listed above, to evaluate changing and/or current levels of phenotypic expression of T2Rs. The level of phenotypic expression of T2Rs may change with age. The embodiments herein may indicate or predict susceptibility to, and clinical course over time of, upper respiratory infections or conditions. For example, embodiments may indicate or predict change in susceptibility or change in clinical course over time as a patient ages. The embodiments herein may indicate or predict susceptibility to, and clinical course over time of, conditions or diseases other than upper respiratory infections or conditions. An embodiment, for example, may include evaluating current levels of phenotypic expression, which may predict susceptibility to, and clinical course of, seasonally occurring infections. The same principles may apply for other chemosensory receptors.

In an embodiment, a method of treatment for a deficit in phenotypic expression of T2Rs, may comprise stimulating T2Rs or other chemosensory receptors by application of an agonist, or plurality of agonists, which may provide increased levels of phenotypic expression. In an embodiment, such increased levels of phenotypic expression may be measurable. In an embodiment such measuring, of increased levels of phenotypic expression, may be performed by, or according to, one or more of the methods of this disclosure. In an embodiment, levels of phenotypic expression may be measured by determining discerned taste response of the human subject. Stimulation of the T2Rs to provide increased levels of phenotypic expression may be acute, or long-term. Long-term stimulation of T2Rs, for example, may increase the level of expression and functionality of T2Rs for many tasters. Individuals with nonfunctional TAS2R38 alleles may have other T2Rs stimulated with one or more agonists to increase their phenotypic expression of T2Rs other than T2R38. Generally, changes in the level of phenotype expression (and taster level) vary with the agonist(s) used, duration of use, and initial phenotype expression.

Stimulation of T2Rs may increase immune response, and one or more agonists may be applied to a human subject to achieve an increased immune response on an acute basis or for a longer term. An acute basis may range from a one-time application to more repeated applications of one or more agonists over, for example, about 12 days. Longer term may be, for example, about 12 days or longer, and may last, for example, for years. These stimulations to increase immune response may employ one or more therapeutic agonists.

Embodiments of this disclosure may include, without limitation:

A) A test method comprising stimulating T2Rs of a human subject with one or more agonists, and detecting one or more products released as a result of stimulation of the T2Rs, wherein the detecting of the products may comprise a method comprising either, or both:

I-i) applying one or more reagents which interact with one or more of the products released because of the stimulation of the T2Rs, and
I-ii) detecting the interaction of the reagent with one or more products, optionally by employing chemiluminescence, an electrochemical sensor, or an optical sensor, to detect the interaction of one or more reagents with one or more of the products, or
II) employing chemiluminescence, an electrochemical sensor, or an optical sensor to detect one or more of the products released because of the stimulation of the T2Rs.
An embodiment of this invention may apply this test method to chemosensory receptors other than T2Rs.

B) The test method as in A) wherein the agonists are separately applied prior to the reagent which interacts with one or more of the products released as a result of stimulation of T2Rs.

C) The test method as in A) wherein the agonist and the reagent that interacts with one or more products released as a result of stimulation of T2Rs are contained on or the same test medium.

D) The test method as in A) wherein the agonists are selected from the group consisting of caffeine, denatonium, strychnine, quinine, terpenes, phenylthiocarbamide, thiourea, sodium benzoate, and any two or more of the foregoing.

E) The test method as in A) wherein the detecting of the products may comprise applying a test medium containing the reagents which interact with the products.

F) The test method as in E) wherein the reagents which interact with the products released as a result of stimulation of T2Rs may comprise a Griess reagent.

G) The test method as in A) wherein the agonist(s) are selected from caffeine, denatonium, strychnine, quinine, terpenes, phenylthiocarbamide, thiourea, sodium benzoate, and any two or more of the foregoing, and wherein the detecting of the products may comprise applying a test medium containing the reagents which interact with the products.

H) The test method as in G) wherein the reagents which interact with the products released as a result of stimulation of T2Rs may comprise a Griess reagent.

I) A test method for determining a level of phenotypic expression of T2Rs, the method comprising

i) stimulating T2Rs by exposing at least a portion of tissue of the human subject to one or more agonists,
ii) recording a discerned level of taste perception by the human subject after the stimulation, and
iii) correlating the discerned level of taste perception to the level of phenotypic expression of the T2Rs.
An embodiment applies the principles of this method to other chemosensory receptors.

J) The method according to I) further comprising repeating steps i)-iii) one or more times, and wherein the agonists may comprise a therapeutic agonist.

K) The method according to I) further comprising repeating steps i)-iii) one or more times to obtain a data set, and optionally performing trend analysis on the data set, wherein the agonists may comprise a therapeutic agonist.

L) The method according to J) wherein the repeating step is performed two or more times, at regular time intervals.

M) The method according to L) wherein the time intervals each are 8 hours, daily, weekly, biweekly, monthly, bimonthly, semiannually, annually, or biannually.

N) The method according to I) wherein the agonists are selected from the group consisting of caffeine, denatonium, strychnine, quinine, terpenes, phenylthiocarbamide, thiourea, sodium benzoate, and any two or more of the foregoing.

O) The method according to J) wherein the therapeutic agonist is selected from the group consisting of caffeine, denatonium, strychnine, quinine, xylitol, grapefruit seed extract or naringenin, a terpene, and any two or more of the foregoing.

P) A method according to I) wherein:

steps i) and ii) are repeated one or more times, and the stimulating by each of one or more different agonists is sequential, the recording of each discerned level of taste perception by the human subject occurs after each stimulation, and the correlating is of one or more of the discerned levels of taste perception to the level of phenotypic expression of the T2Rs of the human subject.

Q) The method according to P) wherein the correlating may comprise employing a computer processor programmed with machine-readable instructions causing the computer processor to:

a) receive and store the discerned levels of taste perception with respect to each agonist,
b) ascribe a weighting to each of the agonists according to their known stimulation of T2Rs,
c) calculate a weighted taste perception from the discerned level of taste perception by multiplying the ascribed weighting and discerned level of taste perception for each agonist applied, then calculating an aggregated, weighted level of taste perception which indicates the level of phenotypic expression.

R) The method according to P) wherein the agonists are selected from the group consisting of caffeine, denatonium, strychnine, quinine, terpenes, phenylthiocarbamide, thiourea, sodium benzoate, and any two or more of the foregoing.

S) A method for evaluating a level of expression or functionality of T2Rs in a human subject, the method comprising stimulating the T2Rs of the human subject with one or more agonists, and detecting one or more products, if any, released as a result of stimulation of the T2Rs by

I-i) applying one or more reagents which interact with one or more of the products released because of the stimulation of the T2Rs, and
I-ii) detecting the interaction of the reagent with one or more products, optionally by employing chemiluminescence, an electrochemical sensor, or an optical sensor, to detect the interaction of one or more reagents with one or more of the products, or
II) employing chemiluminescence, an electrochemical sensor, or an optical sensor to detect one or more of the products released because of the stimulation of the T2Rs, and optionally discerning a level of products produced because of the stimulation of the T2Rs, indicative of level of expression or functionality of the T2Rs, which level of expression or functionality of the T2Rs is correlated to the level of phenotypic expression.

T) The method according to S) wherein the agonists are selected from the group consisting of caffeine, denatonium, strychnine, quinine, terpenes, phenylthiocarbamide, thiourea, sodium benzoate, and any two or more of the foregoing.

U) The method according to S) wherein the detecting of the products may comprise applying a test medium containing one or more reagents which interact with one or more of the products.

V) The method according to U) wherein the reagent which interacts with one or more products released as a result of stimulation of T2Rs may comprise a Griess reagent.

W) The method according to S) wherein the agonist(s) are selected from caffeine, denatonium, strychnine, quinine, terpenes, phenylthiocarbamide, thiourea, sodium benzoate, and any two or more of the foregoing, and wherein the detecting of the products may comprise applying a test medium containing one or more reagents which interact with one or more of the products.

X) The method according to W) wherein one or more of the reagents which interact with one or more products released as a result of stimulation of T2Rs may comprise a Griess reagent.

Y) A test kit comprising at least one test medium containing (a) one or more agonists for T2Rs of a human subject and/or (b) one or more reagents which interact with one or more products released as a result of stimulation of the T2Rs.

Z) The test kit according to Y) wherein the agonists are selected from the group consisting of caffeine, denatonium, strychnine, quinine, terpenes, phenylthiocarbamide, thiourea, sodium benzoate, and any two or more of the foregoing.

Components referred to by chemical name or formula anywhere in the specification or claims hereof, whether referred to in the singular or plural, are identified as they exist prior to coming into contact with another substance referred to by chemical name or chemical type (e.g., another component, a solvent, or etc.). It matters not what chemical changes, transformations and/or reactions, if any, take place in the resulting mixture or solution as such changes, transformations, and/or reactions are the natural result of bringing the specified components together under the conditions called for pursuant to this disclosure. Thus the components are identified as ingredients to be brought together in connection with performing a desired operation or in forming a desired composition. Also, even though the claims hereinafter may refer to substances, components and/or ingredients in the present tense (“comprises”, “is”, etc.), the reference is to the substance, component or ingredient as it existed at the time just before it was first contacted, blended or mixed with one or more other substances, components and/or ingredients in accordance with the present disclosure. The fact that a substance, component or ingredient may have lost its original identity through a chemical reaction or transformation during the course of contacting, blending or mixing operations, if conducted in accordance with this disclosure and with ordinary skill of a chemist, is thus of no practical concern. The invention may comprise, consist, or consist essentially of the materials and/or procedures recited herein.

As used herein, the term “about” modifying the quantity of an ingredient in the compositions, or employed in the methods, of the embodiments refers to variation in the numerical quantity that may occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term about also encompasses amounts that differ due to different equilibrium conditions for a composition resulting from a particular initial mixture. Whether or not modified by the term “about”, the claims include equivalents to the quantities.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

Embodiments are susceptible to considerable variation in practice. Therefore the foregoing description is not intended to limit, and should not be construed as limiting, the embodiments to the particular exemplifications presented hereinabove.

Apparatus, methods and systems according to embodiments of the disclosure are described. Although specific embodiments are illustrated and described herein, it will be appreciated by those of ordinary skill in the art that any arrangement which is calculated to achieve the same purposes can be substituted for the specific embodiments shown. This application is intended to cover any adaptations or variations of the embodiments and disclosure. For example, although described in terminology and terms common to the field of art, exemplary embodiments, systems, methods and apparatus described herein, one of ordinary skill in the art will appreciate that implementations can be made for other fields of art, systems, apparatus or methods that provide the required functions. The invention should therefore not be limited by the above-described embodiments, methods, and examples, but by all embodiments and methods within the scope and spirit of the invention.

In particular, one of ordinary skill in the art will readily appreciate that the names of the methods and apparatus are not intended to limit embodiments or the disclosure. Furthermore, additional methods, steps, and apparatus can be added to the components, functions can be rearranged among the components, and new components to correspond to future enhancements and physical devices used in embodiments can be introduced without departing from the scope of embodiments and the disclosure. One of skill in the art will readily recognize that embodiments are applicable to future systems, future apparatus, future methods, and different materials.

All methods described herein can be performed in a suitable order unless otherwise indicated herein or otherwise clearly contradicted by context. The use of any and all examples, or exemplary language (e.g., “such as”), is intended merely to better illustrate the disclosure and does not pose a limitation on the scope of the disclosure unless otherwise claimed. No language in the specification should be construed as indicating any non-claimed element as essential to the practice of the disclosure as used herein. Terminology used in the present disclosure is intended to include all environments and alternate technologies that provide the same functionality described herein.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. A method of treating a human subject by stimulating chemosensory receptors to increase level of phenotypic expression of the chemosensory receptors, said method comprising:
detecting phenotypic expression deficit by:
first introducing to the chemosensory receptors a first agonist capable of first stimulating the chemosensory receptors by first binding thereto;
detecting a first level of said first stimulating;
identifying a first deficit of said first level in relation to a first reference level, said first deficit being an instance of said phenotypic expression deficit; and
reducing said phenotypic expression deficit by:
selecting a second agonist in relation to said first deficit;
second introducing to the chemosensory receptors said second agonist capable of second stimulating the chemosensory receptors by second binding thereto, said second stimulating providing a reduced phenotypic expression deficit.

28. The method according to claim 27, said method further comprising: the chemosensory receptors comprising T2Rs.

29. The method according to claim 28, said method further comprising:
detecting said reduced phenotypic expression deficit.

30. The method according to claim 29, said method further comprising:
said detecting said reduced phenotypic expression deficit comprising:
detecting a second level of said second stimulating;
identifying a second deficit of said second level in relation to a second reference level, said second deficit being an instance of said reduced phenotypic expression deficit.

31. The method according to claim 30, said method further comprising:
said second reference level equal to said first reference level.

32. The method according to claim 28, said method further comprising:
said detecting said first level further comprising:
recording taste discerned by the human subject.

33. The method according to claim 32, said method comprising:
said first agonist comprising a first plurality of unique agonists;
said detecting said first level further comprising:
recording taste discerned by the human subject in relation to said first plurality of unique agonists in relation to said first stimulating.

34. (canceled)

35. The method according to claim 33, said method comprising:
said first introducing further comprising:
sequentially applying said first plurality of unique agonists.

36. The method according to claim 28, said method further comprising:
said detecting said first level further comprising:
detecting a product released in relation to said first stimulating.

37. The method according to claim 36, said method further comprising:
said detecting said product comprising detection by at least one of the following:
chemiluminescence sensing,
electrochemical sensing, and
optical sensing.

38. The method according to claim 36, said method further comprising:
said detecting said product comprising applying a reagent to interact with said product to provide a detectable phenomenon.

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. The method according to claim 28, said method comprising:
said first agonist consisting of a selected one of the following:
caffeine, denatonium, strychnine, quinine, a terpene,
phenylthiocarbamide, thiourea, and sodium benzoate.

60. The method according to claim 28, said method comprising:
said first agonist comprising a first plurality of unique agonists selected from the following:
caffeine, denatonium, strychnine, quinine, a terpene,
phenylthiocarbamide, thiourea, and sodium benzoate.

61. The method according to claim 28, said method comprising:
said first agonist comprising a first plurality of unique agonists introduced after a control in a sequence as follows:
phenylthiocarbamide,
thiourea,
sodium benzoate.

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. The method according to claim 28, said method further comprising:
said second agonist comprising a therapeutic agonist capable of interacting with said T2Rs to improve phenotypic expression.

74. (canceled)

75. (canceled)

76. (canceled)

77. (canceled)

78. The method according to claim 28, said method further comprising:
administering, subsequent to reducing said phenotypic expression deficit, a third agonist antagonizing the T2Rs characterized by said reduced phenotypic expression deficit to induce phenotypic expression comprising an innate immune response addressing a respiratory infection condition of the human subject, said respiratory infection unaddressed by innate immune response of the T2Rs characterized by unrehabilitated phenotypic expression deficit preceding said reducing said phenotypic expression deficit.

79. The method according to claim 78, said method further comprising:
said administering further comprising:
third selecting said third agonist in relation to one of said reduced phenotypic expression deficit and said innate immune response;
third introducing to the chemosensory receptors said third agonist capable of third stimulating the chemosensory receptors by third binding thereto to provide third stimulating said innate immune response.

80. A method of treating a human subject by stimulating chemosensory receptors to determine susceptibility to respiratory infection, said method comprising:
determining susceptibility to respiratory infection in relation to detecting phenotypic expression deficit by:
first introducing to the chemosensory receptors a first agonist capable of first stimulating the chemosensory receptors by first binding thereto;
detecting a first level of said first stimulating;
identifying a first deficit of said first level in relation to a first reference level, said first deficit being an instance of said phenotypic expression deficit.

81. The method according to claim 80, said method further comprising:
said determining susceptibility further comprising:
correlating said phenotypic expression deficit to susceptibility to respiratory infection.

82. A method of treating a human subject by stimulating chemosensory receptors to determine clinical course of respiratory infection, said method comprising:
determining clinical course of respiratory infection in relation to detecting phenotypic expression deficit by:
first introducing to the chemosensory receptors a first agonist capable of first stimulating the chemosensory receptors by first binding thereto;
detecting a first level of said first stimulating;
identifying a first deficit of said first level in relation to a first reference level, said first deficit being an instance of said phenotypic expression deficit.

83. The method according to claim 82, said method further comprising:
said determining clinical course of respiratory infection further comprising:
correlating said phenotypic expression deficit to susceptibility to respiratory infection.

84. A method for stratification of a patient into a category based on stimulating chemosensory receptors, said method comprising:
introducing to the chemosensory receptors an agonist capable of stimulating the chemosensory receptors when bound thereto;
detecting a level of said stimulating;
identifying a category by comparing said level of said stimulating to at least one reference level of stimulating; and
stratifying the patient into a category in relation to said comparing.

85. A method according to claim 84, said method further comprising:
said reference level being at least one of the following:
a high reference level for homozygous two functional alleles,
a middle reference level for heterozygous one functional allele and one nonfunctional allele, and
a low reference level for homozygous two nonfunctional alleles; and
said category comprising inferred genotype.

86. A method according to claim 84, said method further comprising:
said reference level being at least one of the following:
low level for low phenotypic expression,
moderate level for moderate phenotypic expression,
high level for high phenotypic expression; and
said category comprising inferred level of phenotypic expression.

87. A method according to claim 84, said method further comprising:
said reference level being at least one of the following:
low capacity for treatment by stimulating chemosensory receptors,
moderate capacity for treatment by stimulating chemosensory receptors,
high capacity for treatment by stimulating chemosensory receptors; and
said category being a selected one of the following:
inferred risk of infection,
inferred course of disease prediction,
inferred capacity for treatment,
inferred treatment outcome prediction,
course of treatment, and
inferred level of functionality of chemosensory receptors.

88. A method according to claim 84, said method further comprising:
said reference level being at least one of the following:
Super tasters,
Tasters, and
Non-Tasters; and
said category being a selected one of the following:
inferred risk of infection,
inferred course of disease prediction,
inferred capacity for treatment,
inferred treatment outcome prediction,
inferred course of treatment, and
inferred level of functionality of chemosensory receptors.

89. (canceled)

90. (canceled)

91. A method according to claim 80, said method further comprising:
correlating level of phenotypic expression with risk of infection from SARS-CoV-2.

92. A method according to claim 80, said method further comprising:
correlating level of phenotypic expression with clinical course of disease, SARS-CoV-2.

93. A method according to claim 80, said method further comprising:
correlating level of phenotypic expression with innate immune fitness.

94. (canceled)

95. A method according to claim 84, said method further comprising:
said reference levels being stratified into ranges indicative of innate antimicrobial activity.

96. (canceled)

97. (canceled)
